(19) United States
(12) Patent Application Publication
(10) Pub. No.: US 2009/0060861 A1
(43) Pub. Date: Mar. 5, 2009

(54) Stabilized Polypeptide Formulations

(75) Inventor: Christian Poulsen, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO Nordisk A/S, Bagsvaerd (DK)

(21) Appl. No.: 11/914,564

(22) PCT Filed: May 22, 2006
(86) PCT No.: PCT/EP2006/062490

§ 371 Date: Jun. 6, 2008

(30) Foreign Application Priority Data
May 25, 2005 (EP) 05104481.6

(51) Int. Cl. A61K 38/20; A61K 38/28; A61P 3/10; A61K 38/27; A61K 38/26

(52) U.S. Cl. 424/85.2; 514/3; 514/12

(57) ABSTRACT

The present invention relates to a pharmaceutical formulation comprising a polypeptide and a buffer selected from the group consisting of diethylmalonic acid, trimellitic acid, shikimic acid, glycinamid, 2-amino-2-methyl-1,3-propanediol (AMPD) and tetraethylammonium (T.E.A.) or salts thereof. Further more the invention relates to a method for improving stability of a polypeptide in a purification process comprising the step of applying a buffer selected from the group consisting of diethylmalonic acid, trimellitic acid, shikimic acid, glycinamid, AMPD and T.E.A. or salts thereof to said purification process.

### FIELD OF THE INVENTION

The present invention relates to stable pharmaceutical formulations comprising a polypeptide and a buffer and to a method for improving the stability of a polypeptide in a purification process.

### BACKGROUND OF THE INVENTION

Polypeptide instability during storage or production of pharmaceutical formulations as well as during purification processes is a well-known problem. Among the various physical and chemical deterioration pathways, polypeptide aggregation is probably the most troubling manifestation of polypeptide instability. Aggregation of polypeptides may occur by physical association of two or more polypeptide molecules without any changes in primary structure (physical instability) or by formation of new covalent bond(s) (chemical instability). Physical instability in form of amyloid formation or fibrillation (i.e. formation of insoluble biological inactive aggregates) can cause significant problems for production and storage of polypeptide pharmaceuticals and methods for reduction or elimination of this problem are of considerable interest to the pharmaceutical industry. Additionally, aggregated polypeptides usually exhibit either reduced or in many cases no biological activity, and some aggregates are even believed to be immunogenic or toxic. Fibrillation might cause a significant increase in viscosity; hence this phenomenon is sometimes also referred to as gelation.

The addition of buffers to polypeptide-containing pharmaceutical solutions is essential in order to stabilize pH during purification or of the pharmaceutical solution within a desired pH range. Conventionally, phosphate buffers have been used as the preferred buffering agent for pharmaceutical formulations containing polypeptides.

### SUMMARY OF THE INVENTION

It has been found that pharmaceutical polypeptide formulations having increased physical stability can be obtained by adding certain buffer compounds or salts thereof to said formulations.

Thus in one aspect the present invention is related to a pharmaceutical formulation comprising a polypeptide and a buffer selected from the group consisting of diethylmalonic acid (diethylpropanedioic acid), trimellitic acid (1,2,4-benzenetricarboxylic acid), shikimic acid ([3R-(3α,4α,5β)]-3,4,5-trihydroxy-1-cyclohexene-1-carboxylic acid), glycinamid, AMPD (2-amino-2-methyl-1,3-propanediol) and T.E.A. (tetraethylammonium) or salts thereof.

In one embodiment of the present invention the buffer is selected from the group consisting of diethylmalonic acid, trimellitic acid and glycinamid or salts thereof.

In another embodiment of the present invention the buffer is selected from the group consisting of shikimic acid, 2-amino-2-methyl-1,3-propanediol (AMPD) and tetraethylammonium (T.E.A.) or salts thereof.

In another embodiment of the present invention the buffer is diethylmalonic acid or a salt thereof.

In another embodiment of the present invention the buffer is trimellitic acid or a salt thereof.

In another embodiment of the present invention the buffer is shikimic acid or a salt thereof.

In another embodiment of the present invention the buffer is glycinamid or a salt thereof.

In another embodiment of the present invention the buffer is 2-amino-2-methyl-1,3-propanediol (AMPD) or a salt thereof.

In another embodiment of the present invention the buffer is tetraethylammonium (T.E.A.) or a salt thereof.

In another aspect the invention is related to a method for improving the stability of a polypeptide in a purification process. The method will comprise addition of an adequate amount of a buffer selected from the group consisting of diethylmalonic acid (diethylpropanedioic acid), trimellitic acid (1,2,4-benzenetricarboxylic acid), shikimic acid ([3R-(3α,4α,5β)]-3,4,5-trihydroxy-1-cyclohexene-1-carboxylic acid), glycinamid, AMPD (2-amino-2-methyl-1,3-propanediol) and T.E.A. (tetraethylammonium) or salts thereof.

### BRIEF DESCRIPTION OF THE DRAWINGS

FIG. 1

Physical stability of polypeptide (insulin aspart) compositions containing diethylmalonic acid or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 2

Physical stability of polypeptide (insulin aspart) compositions containing trimellitic acid or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 3

Physical stability of polypeptide (insulin aspart) compositions containing shikimic acid or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 4

Physical stability of polypeptide (insulin aspart) compositions containing glycinamid hydrochloride or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 5

Physical stability of polypeptide (insulin aspart) compositions containing AMPD (2-amino-2-methyl-1,3-propanediol) or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 6

Physical stability of polypeptide (insulin aspart) compositions containing T.E.A. chloride (tetraethylammonium chloride) or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 7

Physical stability of polypeptide (human insulin) compositions containing diethylmalonic acid (legend: DIE), trimellitic acid (legend: TRI), shikimic acid (legend: SHI), glycinamid hydrochloride (legend: GLY), 2-amino-2-methyl-1,3-propanediol (legend: AMPD), tetraethylammonium chloride (legend: TEA) or phosphate. The figure shows the relative lag-time (a measure of physical instability). The results are expressed as the average relative lag-time of each composition (to ease comparison, data normalized to give phosphate buffer index 100). Error bars indicate SEM, n=12.

FIG. 8

Physical stability of polypeptide (acylated insulin derivate B29-N^{ε}-tetradecanoyl-des(B30)-human insulin) containing diethylmalonic acid (legend: DIE), trimellitic acid (legend: TRI), shikimic acid (legend: SHI), glycinamid hydrochloride (legend: GLY), 2-amino-2-methyl-1,3-propanediol (legend: AMPD), tetraethylammonium chloride (legend: TEA) or phosphate. The figure shows the relative lag-time (a measure of physical instability). The results are expressed as the average relative lag-time of each composition (to ease comparison, data normalized to give phosphate buffer index 100). Error bars indicate SEM, n=12.

### DESCRIPTION OF THE INVENTION

It has been found that pharmaceutical polypeptide formulations having increased stability can be obtained by adding to said formulation a buffer selected from the group consisting of diethylmalonic acid (diethylpropanedioic acid), trimellitic acid (1,2,4-benzenetricarboxylic acid), shikimic acid ([3R-(3α,4α,5β)]-3,4,5-trihydroxy-1-cyclohexene-1-carboxylic acid), glycinamid hydrochloride, AMPD (2-amino-2-methyl-1,3-propanediol) and T.E.A. chloride (tetraethylammonium chloride) or salts thereof.

The term “stabilized solution” refers to a polypeptide solution with increased physical stability, increased chemical stability or increased physical and chemical stability. Pharmaceutical formulations and solutions of polypeptides from various processing steps (eg. a purification step) are examples of such solutions.

The term “physical stability” of the protein solution as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein solution is evaluated by means of visual inspection and/or turbidity measurements after exposing the solution filled into suitable containers (e.g. cartridges or vials) to mechanical/physical stress (e.g. agitation) at different temperatures for various time periods. Visual inspection of the solutions is performed in a sharp focused light with a dark background. The turbidity of the solution is characterized by a visual score ranking the degree of turbidity for instance on a scale from 0 to 3 (a solution showing no turbidity corresponds to a visual score 0, and a solution showing visual turbidity in daylight corresponds to visual score 3). A solution is classified physically unstable with respect to protein aggregation, when it shows visual turbidity in daylight. Alternatively, the turbidity of the solution can be evaluated by simple turbidity measurements well-known to the skilled person. Physical stability of the aqueous protein solution can also be evaluated by using a spectroscopic agent or probe of the conformational status of the protein. The probe is preferably a small molecule that preferentially binds to a non-native conformer of the protein. One example of a small molecular spectroscopic probe is Thioflavin T.

The term “chemical stability” of the protein solution as used herein refers to chemical covalent changes in the protein structure leading to formation of chemical degradation products with potentially less biological potency and/or potentially increased immunogenic properties compared to the native protein structure.

Hence, as outlined above, a “stabilized polypeptide solution” refers to a solution with increased physical stability, increased chemical stability or increased physical and chemical stability. Further, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

The stabilization is achieved by addition of the selected group of buffers to pharmaceutical formulation or solution comprising the polypeptide in question. The optimal concentration of the buffer to obtain an adequate chemical stabilization of a certain polypeptide depends on various parameters such as the buffer, the polypeptide concentration and structure.

Thus the concentration of the buffer may vary within a relatively large range depending on the polypeptide in question, the specific buffer and the other constituents in the pharmaceutical formulation. The concentration of the buffer will typically be in the range from 0.01-100 mM.

In another embodiment the concentration of the buffer is between 1 and 50 mM or between 3 and 25 mM.

In a further embodiment the concentration of the buffer is between 3 and 20 mM, between 4 and 20 mM, between 5 and 20 mM, between 5 and 18 mM, between 5 and 17 mM, between 5 and 16 mM.

The optimal buffer concentration may vary slightly between the relevant buffers according to the invention.

The pH of the pharmaceutical formulation may be in the range from about 2 to about 10 but will typically be in the range from about 4 to about 8.5.

In a further embodiment the pH will be from 4.5-6.5, from 5.5.-6.5, from 6.5-9, from 6.5-8.5 or from 7-8.

One object of the present invention is to provide a pharmaceutical formulation comprising a polypeptide compound which is present in a concentration from 0.01 mg/ml to about 100 mg/ml, wherein said formulation has a pH from 2.0 to about 10.0 and wherein the formulation comprises a buffer selected from the group consisting of diethylmalonic acid, trimellitic acid, shikimic acid, glycinamid, 2-amino-2-methyl-1,3-propanediol (AMPD) and tetraethylammonium (T.E.A.) or salts thereof.

Another object of the present invention is to provide a pharmaceutical formulation comprising a polypeptide compound which is present in a concentration from 0.01 mg/ml to about 100 mg/ml, wherein said formulation has a pH from 2.0 to about 10.0 and wherein the formulation comprises a buffer selected from the group consisting of diethylmalonic acid, trimellitic acid and glycinamid or salts thereof.

Another object of the present invention is to provide a pharmaceutical formulation comprising a polypeptide compound which is present in a concentration from 0.01 mg/ml to about 100 mg/ml, wherein said formulation has a pH from 2.0 to about 10.0 and wherein the formulation comprises a buffer selected from the group consisting of shikimic acid, 2-amino-2-methyl-1,3-propanediol (AMPD) and tetraethylammonium (T.E.A.) or salts thereof.

In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the health care provider or the patient adds solvents and/or diluents prior to use.

In another embodiment the pharmaceutical formulation is a dried formulation (e.g. freeze-dried or spray-dried) ready for use without any prior dissolution.

The pharmaceutical formulation may further comprise one or more conventional buffers or buffer systems. Such a conventional buffer may be selected from the group consisting of sodium acetate, sodium carbonate, citric acid, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinic acid, maleic acid, fumaric acid, tartaric acid, aspartic acid and/or mixtures and/or salts thereof. Particularly such conventional buffer or buffer system comprise a phosphate buffer.

In one embodiment the total concentration of buffers is in the range from 0.01-100 mM, particularly the total concentration of the buffer is at most 50 mM, such as 1-50 mM.

In one embodiment the pharmaceutical formulation further comprise components selected from the group consisting of stabilizer(s), amino acid base(s), antimicrobial preservative(s), chelating agent(s), surfactant(s) and tonicity agent(s).

The use of a stabilizer in pharmaceutical compositions is well-known to the skilled person. For convenience reference is made to Remington: The Science and Practice of Pharmacy, 19^{th} edition, 1995. In one embodiment the compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, or nithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention, methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freezethawing or mechanical shearing.

In one embodiment the compositions further comprise one or more antimicrobial preservatives. Suitable pharmaceutically acceptable preservatives may be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thimerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. Isotonic agents may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propylene glycol), 1,3-propanediol, 1,3-butanediol) polyethylene glycol (e.g. PEG400), or mixtures thereof. Any sugar such as mono-, di-, or polysaccharides, or watersoluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. Chelating agents may be selected from salts of ethylenediamine tetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience, reference is made to Remington: The Science and Practice of Pharmacy, 19^{th} edition, 1995.

In a further embodiment of the invention the formulation further comprises a surfactant. Surfactants may be selected from a detergent, ethoxylated castor oil, polyglycolyzed glycerides, acetylated monoglycerides, sorbitan fatty acid esters, polyoxypropylenepolyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)- derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

In a further embodiment of the invention the formulation further comprises protease inhibitors such as EDTA (ethylenediamine tetraacetic acid) and benzamidine hydrochloride, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

In one embodiment of the invention the pharmaceutical formulation comprises a polypeptide selected from the group comprising insulin, human growth hormone, glucagon, GLP-1, exendin-4, FVII, FXIII, a mixture of FVII and FXIII, IL-20, IL-21, IL-28a, IL-29, IL-31 and/or analogues and/or derivates thereof.

Other suitable polypeptides may be selected among the group consisting of ACTH, corticotropin-releasing factor, angiotensin, calcitonin, IGF-1, IGF-2, enterogastrin, somatostatin, somatotropin, somatomedin, parathyroid hormone, thrombopoietin, erythropoietin, hypothalamic releasing factors, prolactin, thyroid stimulating hormones, endorphins, enkephalins, vasopressin, oxytocin, opioids and analogues thereof, superoxide dismutase, interferon, asparaginase, arginase, arginine deaminase, adenosine deaminase and ribonuclease.

In one embodiment of the invention the polypeptide is insulin. In pharmaceutical formulations of insulin, the insulin molecule exists in an equilibrium between insulin monomers, insulin dimers and insulin hexamers and fibrillation of insulin is believed to occur through the insulin monomer (in form of a partially unfolded insulin monomer intermediates).

As the presence of zinc ions shifts the equilibrium towards the hexameric form, which in turn stabilizes the formulation against fibrillation, zinc ions are often added to marketed insulin formulations with the purpose of stabilizing the formulation. However, as many insulin formulations contain phosphate buffer (for stabilizing pH) and phosphate binds zinc ions (potentially leading to precipitation of zinc phosphate complexes), a delicate equilibrium exists between zinc ions bound in the core of the insulin hexamer and zinc ions bound to phosphate. The present invention provides polypeptide formulations with increased stability in comparison with the known phosphate containing pharmaceutical formulations.

Accordingly, in a particular embodiment of the invention the polypeptide is insulin. Insulin is a polypeptide consisting of two amino acid chains: An A chain and a B chain connected to one another by means of two disulfide bridges. Insulin can be divided into naturally occurring insulin, insulin analogues and insulin derivates but the definitions are not mutually exclusive and various molecules can meet more than one of the definitions.

Naturally occurring insulin refers to mammalian insulin—i.e. insulin molecules obtained from or identical to the insulin molecules from mammalian sources (eg. human, bovine or porcine). The A chain of naturally occurring insulin consists of 21 amino acids and the B chain of naturally occurring insulin consist of 30 amino acids. Naturally occurring insulin can be produced by extraction from pancreatic glands or by recombinant DNA techniques in various host cells.

In one embodiment the pharmaceutical formulation according to the invention is a formulation wherein the polypeptide is an insulin derivate or an insulin analogue.

Insulin analogues are analogues of naturally occurring insulin, namely human insulin or animal insulin, which differ by substitution of at least one naturally occurring amino acid residue with other amino acid residues and/or addition/deletion of at least one amino acid residue from the corresponding, otherwise identical, naturally occurring insulin. The added amino acid residues can also be those which do not occur naturally. Examples of insulin analogues are analogues of human insulin where the amino acid residue at position B28 is Asp, Lys, Leu, Val, or Ala and position B29 is Lys or Pro; or B3 is Lys and B29 is Glu; or A21 is Gly and Arg has been added to B31 and B32; or where the amino acid residues in B28-B30 have been deleted; or where the amino acid residue at B27 has been deleted; or where the amino acid residue at B30 has been deleted.

Insulin derivates are derivates of naturally occurring insulin or insulin analogues in which at least one organic molecule is bound to one or more of the amino acid residues. Examples of insulin derivates are derivates of naturally occurring insulin or insulin analogues where the organic molecule bound to the amino acid residues is a lipophilic molecule. Examples of insulin derivates where the organic molecule bound to the amino acid residues is a lipophilic molecule are B29-N^{ε}-tetradecanoyl-des(B30)-human insulin, B29-N^{ε}-tetradecanoylhuman insulin, B29-N^{ε}-hexadecanoyl human insulin, B28-N^{ε}-tetradecanoyl-Lys^{B28}Pro^{B29} human insulin, B28-N^{ε}-hexadecanoyl-Lys^{B28}Pro^{B29} human insulin, B30-N^{ε}-tetradecanoylThr^{B29}Lys^{B30} human insulin, B30-N^{ε}-hexadecanoyl-Thr^{B29}Lys^{B30}-human insulin, B29-N^{ε}-(Nhexadecanoyl-γ-glutamyl)-des(B30)-human insulin, B29-N^{ε}-(N-litocholyl-γ-glytamyl)-des(B30) human insulin, B29-N^{ε}-(ω-carboxyheptadecanoyl)-des(B30)-human insulin, N^{ε}B29-tetradecanoyl des(B30) human insulin, N^{ε}B28-tetradecanoyl Lys^{B28}Pro^{B29} human insulin, N^{ε}B29-tetradecanoyl Asp^{B28} human insulin and Lys^{B29} (N^{ε}-hexadecandioyl-γ-Glu)-des(B30) human insulin.

In another embodiment the pharmaceutical formulation according to the invention is a formulation wherein the polypeptide is an Asp^{B28} analogue of human insulin.

In another embodiment the pharmaceutical formulation according to the invention is a formulation wherein the polypeptide is B29-N^{ε}-tetradecanoyl-des(B30)-human insulin.

In another embodiment the pharmaceutical formulation according to the invention is a formulation wherein the polypeptide is B29-N^{ε}-(N-litocholyl-γ-glytamyl)-des(B30)-human insulin.

In another embodiment the pharmaceutical formulation according to the invention is a formulation wherein the polypeptide is Lys^{B29} (N^{ε}-hexadecandioyl-γ-Glu)-des(B30) human insulin.

It has been demonstrated that the presence of the buffers according to the invention increase the stability of a polypeptide compared to the presence of 7 mM phosphate buffer.

In another embodiment the invention relates to a method for improving stability of a polypeptide during processing such as a purification process. The inventive method comprises the step of applying a buffer or a combination of buffers selected from the group consisting of diethylmalonic acid, trimellitic acid, shikimic acid, glycinamid hydrochloride, AMPD and T.E.A. chloride or salts thereof to the solution containing the polypeptide to be purified.

The method will comprise addition of an adequate amount of the buffer or a salt thereof to the solution containing the polypeptide to be purified. The buffer will typically be added in the last 2 or 3 purification steps (the polishing steps). The purification steps may be ion exchange chromatography, HPLC chromatography, ultrafiltration or diafiltration or other buffer exchange processes.

### EXAMPLES

### Example 1

Aqueous solutions containing insulin aspart (Asp^{B28} human insulin) were prepared by mixing sub-solutions, containing the individual components (including the buffer component), followed by pH-adjustment by addition of diluted hydrochloric acid or sodium hydroxide to give compositions as displayed in Table 1.

TABLE 1
Example of Composition

| Component | Concentration | Main Function |
|---|---|---|
| Asp^{B28} human insulin | 600 | Active Ingredient |
| (nmol/mL) | | |
| Zinc (μg/mL) | 19.6 | Stabilizer |
| Phenol (mg/mL) | 1.50 | Preservative Agent |
| m-Cresol (mg/mL) | 1.72 | Preservative Agent |
| Glycerol (mg/mL) | 16 | Tonicity Agent |
| Sodium chloride (mg/mL) | 0.58 | Tonicity/Stabilizing Agent |
| Buffer (mM) | Varies | Buffer Agent |
| pH | 7.4 | — |

Compositions comprising the buffer components covered by the present invention were prepared according to the above-mentioned and a reference composition containing 7 mM sodium phosphate as buffer component was prepared in parallel.

The physical stability (i.e. the tendency towards formation of fibrils) of the compositions was assessed using a Thioflavin T (ThT) assay. The principle behind the ThT-assay is based on the fluorescence characteristics of ThT, which in the free state (i.e. not bound to fibrils) fluoresces faintly with excitation and emission maxima of 350 and 438 nm, respectively. However, in the presence of fibrils, ThT exhibits a new excitation maximum at 450 nm and enhanced emission at 482 nm.

Samples of each composition was mixed with an aqueous ThT-solution (0.1 mM ThT) in a volumetric ratio of 975:25 and twelve samples of each mixture were transferred to a microtiter plate. The microtiter plate was placed in a Fluoroskan Ascent FL reader and the samples were subjected to conditions known to accelerate the fibril formation process (for instance: 37° C., shake: 1200 rpm, 1 mm amplitude). Fluorescence (λ_{Ex=444 nm}; λ_{Em=485 nm}) in each plate well was measured for up to 15 hours (5 minutes intervals). Fluorescence vs. time plots were generated and the time until an increase in fluorescence was observed (herein defined as Lag-Time) was estimated as the intercept between linear approximation of the Lag Zone and Fibrillation Zone. An increase in lag-time corresponds to an increased physical stability. Twelve lag-time observations were obtained for each composition.

### Example 2

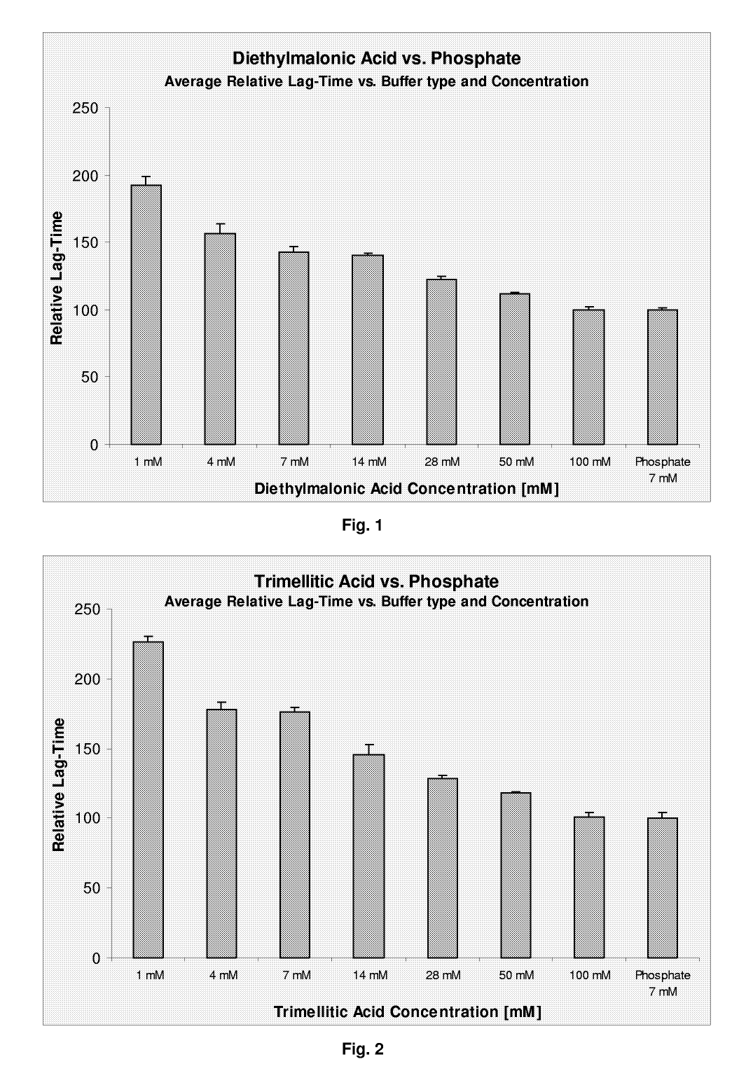

Compositions comprising 1-100 mM diethylmalonic acid or 7 mM phosphate as buffer components were prepared following example 1. The physical stability of the compositions was examined according to example 1. The polypeptide stability was increased in compositions comprising diethylmalonic acid compared to compositions comprising phosphate buffer (7 mM). The results are presented in FIG. 1. No significant impact on chemical stability of the polypeptide was observed, as measured by the amount of degradation products formed during 3 months storage at 5° C. or 37° C. (not shown).

### Example 3

Compositions comprising 1-100 mM trimellitic acid or 7 mM phosphate as buffer components were prepared following example 1. The physical stability of the compositions was examined according to example 1. The polypeptide stability was increased in compositions comprising trimellitic acid compared to compositions comprising phosphate buffer (7 mM). The results are presented in FIG. 2. No significant impact on chemical stability of the polypeptide was observed, as measured by the amount of degradation products formed during 3 months storage at 5° C. or 37° C. (not shown).

### Example 4

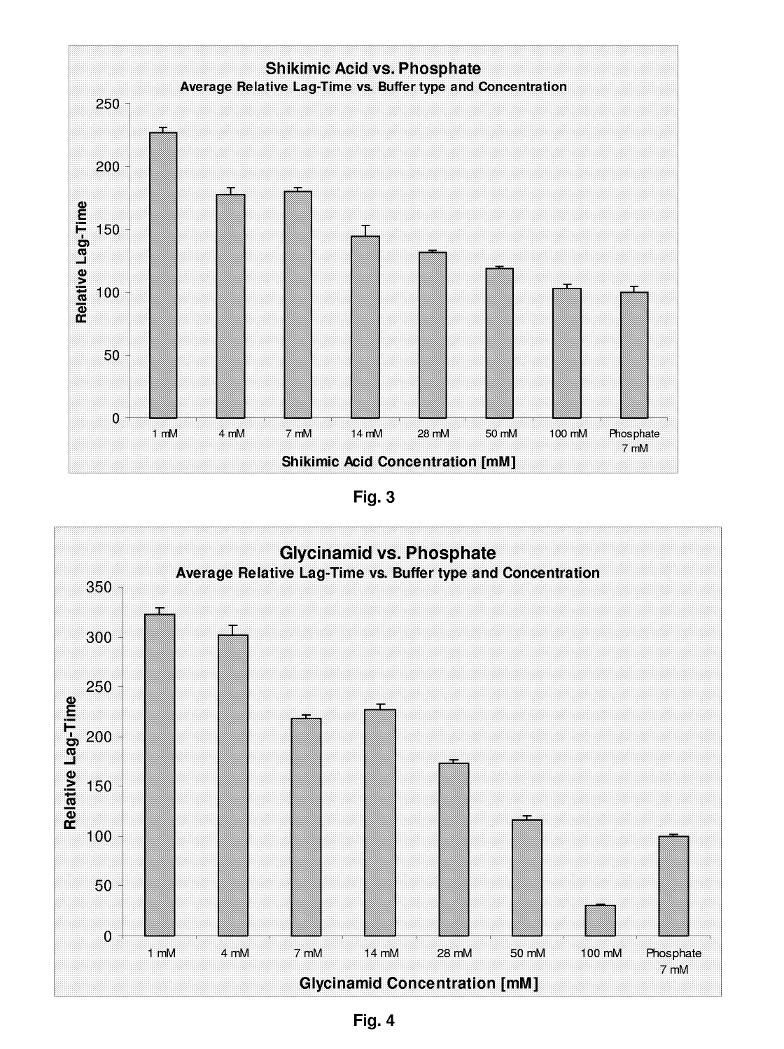

Compositions comprising 1-100 mM shikimic acid or 7 mM phosphate as buffer components were prepared following example 1. The physical stability of the compositions was examined according to example 1. The polypeptide stability was increased in compositions comprising shikimic acid compared to compositions comprising phosphate buffer (7 mM). The results are presented in FIG. 3. No significant impact on chemical stability of the polypeptide was observed, as measured by the amount of degradation products formed during 3 months storage at 5° C. or 37° C. (not shown).

### Example 5

Compositions comprising 1-100 mM glycinamid hydrochloride or 7 mM phosphate as buffer components were prepared following example 1. The physical stability of the compositions was examined according to example 2. The polypeptide stability was increased in compositions comprising glycinamid hydrochloride (below 100 mM) compared to compositions comprising phosphate buffer (7 mM). The results are presented in FIG. 5. No significant impact on chemical stability of the polypeptide was observed, as measured by the amount of degradation products formed during 3 months storage at 5° C. or 37° C. (not shown).

### Example 6

Compositions comprising 1-100 mM AMPD (2-amino-2-methyl-1,3-propanediol) or 7 mM phosphate as buffer components were prepared following example 1. The physical stability of the compositions was examined according to example 1. The polypeptide stability was increased in compositions comprising AMPD compared to compositions comprising phosphate buffer (7 mM). The results are presented in FIG. 5. No significant impact on chemical stability of the polypeptide was observed, as measured by the amount of degradation products formed during 3 months storage at 5° C. or 37° C. (not shown).

### Example 7

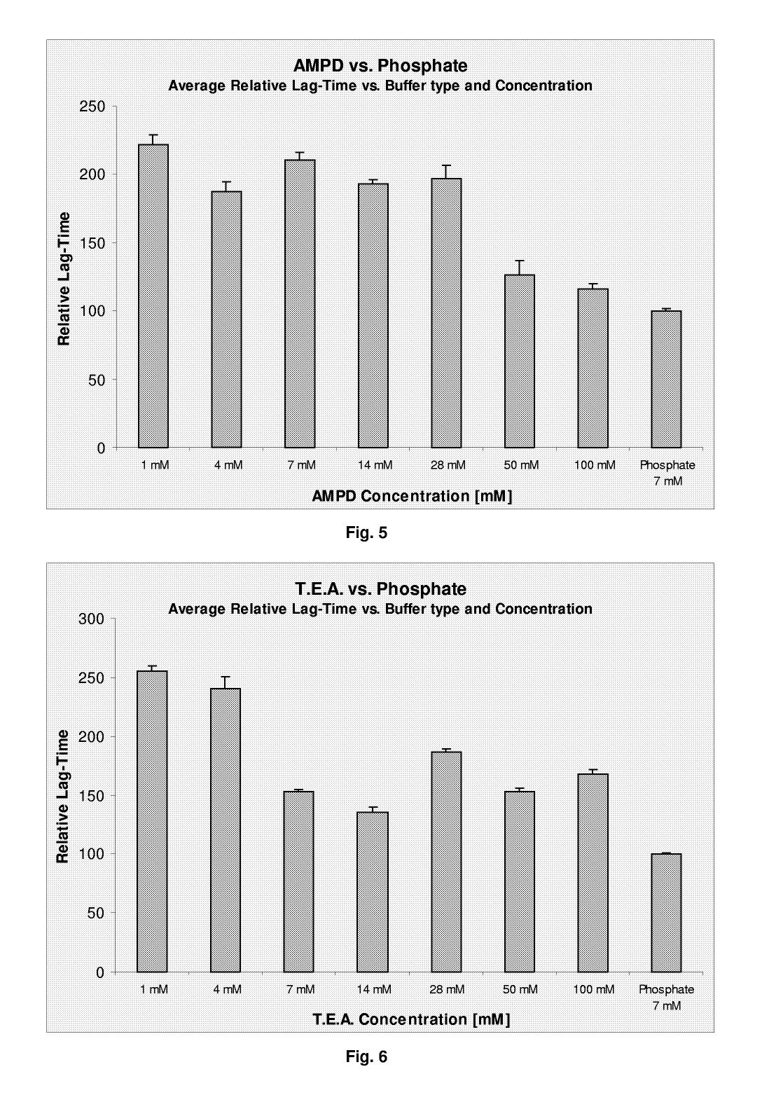

Compositions comprising 1-100 mM T.E.A. chloride (tetraethylammonium chloride) or 7 mM phosphate as buffer components were prepared following example 1. The physical stability of the compositions was examined according to example 1. The polypeptide stability was increased in compositions comprising T.E.A. chloride compared to compositions comprising phosphate buffer (7 mM). The results are presented in FIG. 6. No significant impact on chemical stability of the polypeptide was observed, as measured by the amount of degradation products formed during 3 months storage at 5° C. or 37° C. (not shown).

### Example 8

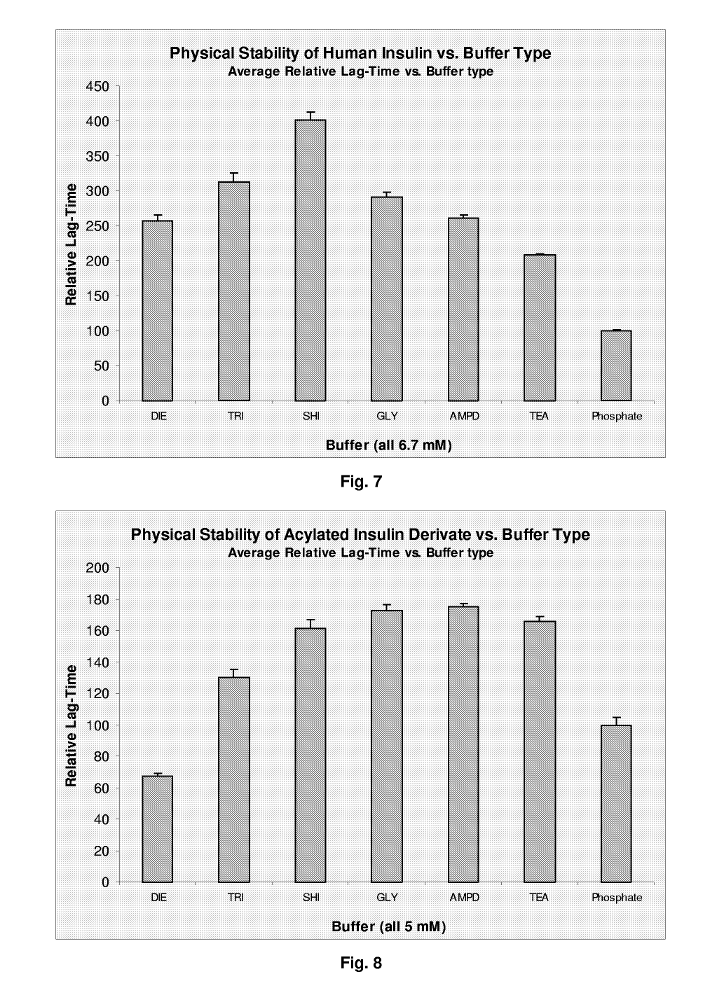

Compositions comprising human insulin and 6.7 mM diethylmalonic acid (legend: DIE), trimellitic acid (legend: TRI), shikimic acid (legend: SHI), glycinamid hydrochloride (legend: GLY), 2-amino-2-methyl-1,3-propanediol (legend: AMPD), tetraethylammonium chloride (legend: TEA) or phosphate were prepared. The physical stability of the compositions was examined according to example 1. The polypeptide stability was increased in compositions comprising diethylmalonic acid, trimellitic acid, shikimic acid, glycinamid hydrochloride, 2-amino-2-methyl-1,3-propanediol, tetraethylammonium chloride compared to compositions comprising phosphate buffer. The results are presented in FIG. 7.

### Example 9

Compositions comprising an acylated insulin derivate B29-N^{ε}-tetradecanoyl-des(B30)-human insulin and 5 mM diethylmalonic acid (legend: DIE), trimellitic acid (legend: TRI), shikimic acid (legend: SHI), glycinamid hydrochloride (legend: GLY), 2-amino-2-methyl-1,3-propanediol (legend: AMPD), tetraethylammonium chloride (legend: TEA) or phosphate were prepared. The physical stability of the compositions was examined according to example 1. The polypeptide stability was increased in compositions comprising trimellitic acid, shikimic acid, glycinamid hydrochloride, 2-amino-2-methyl-1,3-propanediol, tetraethylammonium chloride compared to compositions comprising phosphate buffer. The results are pre-sented in FIG. 8.

1. A pharmaceutical formulation comprising a polypeptide and a buffer or a combination of buffers is selected from the group consisting of diethylmalonic acid, trimellitic acid, shikimic acid, glycinamid, 2-amino-2-methyl-1,3-propanediol (AMPD) and tetraethylammonium (T.E.A.) or salts thereof.

2. A pharmaceutical formulation according to claim 1, wherein the buffer or the combination of buffers is selected from the group consisting of diethylmalonic acid, trimellitic acid and glycinamid or salts thereof.

3. A pharmaceutical formulation according to claim 1, wherein the buffer or the combination of buffers is selected from the group consisting of shikimic acid, 2-amino-2-methyl-1,3-propanediol (AMPD) and tetraethylammonium (T.E.A.) or salts thereof.

4. A pharmaceutical formulation according to claim 1, wherein the concentration of buffer or combination of buffers is in the range from 0.01-100 mM

5. A pharmaceutical formulation according to claim 4, wherein the concentration of buffer or combination of buffers is in the range from 0.1-50 mM.

6. A pharmaceutical formulation according to claim 5, wherein the concentration of buffer or combination of buffers is in the range from 3-25 mM.

7. A pharmaceutical formulation according to claim 6, wherein the concentration of buffer or combination of buffers is in the range from 5-16 mM.

8. A pharmaceutical formulation according to claim 1, wherein the polypeptide is selected from the group comprising of insulin, human growth hormone, glucagon, GLP-1, exendin-4, FVII, FXIII, a mixture of FVII and FXIII, IL-20, IL-21, IL-28a, IL-29, IL-31 or analogues or derivatives thereof.

9. The pharmaceutical formulation according to claim 8, wherein the polypeptide is insulin or an analogue or a derivative thereof.

10. A method for improving the stability of a polypeptide in a purification process or in a pharmaceutical formulation comprising the step of applying a buffer or a combination of buffers selected from the group consisting of diethylmalonic acid, trimellitic acid, shikimic acid, glycinamid, AMPD and T.E.A. or salts thereof to said purification process or pharmaceutical formulation.
